SEQUENCE LISTING (4 sequences)

SEQ ID NO: 1 - protein, 11 aa, Artificial Sequence
  CDR3 of light chains of mAb W108
QQGNTLPVDVR

SEQ ID NO: 2 - protein, 11 aa, Artificial Sequence
  CDR3 of heavy chains of mAb W108
LSLHLLHSPAV

SEQ ID NO: 3 - protein, 41 aa, Dermatophagoides pteronyssinus
FGISNYCQIYPPNVNKIREALAQTHSAIAVIIGIKDLDAFR

SEQ ID NO: 4 - protein, 28 aa, Dermatophagoides pteronyssinus
DNGYQPNYHAVNIVGYSNAQGVDYWIVR

(19) United States
(12) Patent Application Publication
(10) Pub. No.: US 2015/0166644 A1
(43) Pub. Date: Jun. 18, 2015

(54) DERMATOPHAGOID PTERONYSSINUS (DERP1) ANTIGEN EPITOPE AND ANTI-DER P1 ANTIBODY

(71) Applicant: Promd Biotech Co., Ltd., Taipei City (TW)

(72) Inventor: JIU-YAO WANG, TAINAN (TW)

(21) Appl. No.: 14/576,521

(22) Filed: Dec. 19, 2014

Related U.S. Application Data
(62) Division of application No. 13/470,666, filed on May 14, 2012, now abandoned.

(51) Int. Cl. C07K 16/18

(52) U.S. Cl. CPC C07K 16/18; A61K 2039/505

(57) ABSTRACT

The present invention provides a novel isolated peptide segment which is a Dermatophagoid pteronyssinus (Der p1) antigen epitope, comprising one selected from the group consisting of the peptide segments having the amino acid sequences set forth in SEQ ID NO: 3 and SEQ ID NO: 4, and combination thereof. Also provided is an isolated antibody or antigen binding fragment thereof specific for a Dermatophagoid pteronyssinus (Der p1) antigen, which binds to the peptide segments having the amino acid sequences set forth in SEQ ID NO: 3 and SEQ ID NO: 4, and combination thereof. The present invention further provides a pharmaceutical composition for treating an IgE-mediated disease comprising a therapeutically effective amount of the antibody or antigen binding fragment.

### CROSS-REFERENCE TO RELATED APPLICATIONS

This application claims benefit of U.S. Non-Provisional Application No. 13/470,666, filed May. 14, 2012, which is hereby incorporated by reference.

### FIELD OF THE INVENTION

The present invention pertains to Dermatophagoides pteronyssinus (Der p 1) antigen and antibody specific to Der p 1 allergen; in particular, the present invention is directed to an anti-Der p 1 antibody useful for treating allergen-induced related diseases.

### BACKGROUND OF THE INVENTION

House dust mites are a major source of allergens which contribute to the rising incidence of allergic diseases. In Taiwan, about 80% of asthmatic children are sensitive to Dermatophagoides pteronyssinus (Der p 1), depending on their geographic location within Taiwan.

Der p 1 is a 27-36 kDa cysteine protease produced in the mite as an enzymatically inactive pro-enzyme which becomes active after cleavage and detachment of the pro-peptide. The gene encoding Der p1 precursor (P 08176, 320 a.a.) has been cloned and sequenced and displays an iso-allergenic variation. Apart from inhibiting the activity of the proenzyme, the pro-peptide may also act as a folding scaffold for mature Der p 1, as is suggested for other proteases. A recombinant Der p 1 was produced in Escherichia coli, but the resulting protein had much reduced IgE binding activity, indicating improper folding. In Pichia pastoris, a recombinant pro-Der p 1 can be produced as a hyperglycosylated pro-enzyme with reduced enzymatic activity and IgE binding. However, after in vitro maturation, enzymatic activity and IgE binding can be restored independently of glycosylation.

Recently, the structures of the pro-enzyme and the mature form of Der p 1 have been demonstrated by X-ray crystallography and this has mostly confirmed that the original models were constructed from the coordinates of papain. However, exact interactions between the residues within the folds and the epitopes responsible for binding to IgE, which may be important for the clinical therapeutic use of Der p 1, are not known. The majority of monoclonal antibodies raised against Der p 1 do not inhibit the binding of human IgE to Der p 1, indicating that they recognize different epitopes, or are of much lower affinity than human anti-Der p 1 IgE.

### BRIEF SUMMARY OF THE INVENTION

It is expectedly found that an anti-Der p 1 antibody 1 can block its IgE binding epitope and have cysteine protease activity.

The aim of the present invention is to provide an isolated peptide segment which is a Dermatophagoid pteronyssinus (Der p1) antigen epitope, comprising one selected from the group consisting of the peptide segments having the amino acid sequences set forth in SEQ ID NO: 3 and SEQ ID NO: 4, and combination thereof.

In one aspect, an isolated antibody or antigen binding fragment thereof specific for a Dermatophagoid pteronyssinus (Der p1) antigen, which specifically binds to the peptide segments having the amino acid sequences set forth in SEQ ID NO: 3 and SEQ ID NO: 4, and combination thereof.

In another aspect, the invention provides a method for preventing and/or treating an IgE-mediated disease in a subject with a therapeutically effective amount of the antibody or antigen binding fragment.

The present invention also provides a pharmaceutical composition for treating an IgE-mediated disease comprising a therapeutically effective amount of the antibody or antigen binding fragment.

The present invention also features a method for treating and/or preventing an IgE-mediated disease in a subject comprising administering the subject with a therapeutically effective amount of the peptide segment.

It is believed that a person of ordinary knowledge in the art where the present invention belongs can utilize the present invention to its broadest scope based on the descriptions herein with no need of further illustration. Therefore, the following descriptions should be understood as of demonstrative purpose instead of limitative in any way to the scope of the present invention.

### BRIEF DESCRIPTION OF THE SEVERAL VIEWS OF THE DRAWINGS

The foregoing summary, as well as the following detailed description of the invention, will be better understood when read in conjunction with the appended drawings.

For the purpose of illustrating the invention, there are shown in the drawings embodiments which are presently preferred. It should be understood, however, that the invention is not limited to the precise arrangements and instrumentalities shown.

In the drawings:

FIGS. 1A-1C show the competitive binding analysis of monoclonal antibody W108 using patient IgE to Der p allergen. FIG. 1A shows the binding of Der p-sensitive sera and pre-incubated with mAb W108 detected using HRP-mouse anti-human IgE Ab. FIG. 1B shows the mAb W108 had the highest blocking activity and inhibited more than 70% of the allergen-specific IgE binding in the pooled sera of nine Der p-sensitive allergic asthmatic children. FIG. 1C shows the cysteine protease activity of Der p 1 is inhibited by mAb W108.

FIGS. 2A-2B depict MALDI-TOF MS and LC MS/MS profile of tryptic digests of spot 1. FIG. 2A shows spot 1 protein was digested in situ with trypsin. The prominent mass peak for database searches, and the protein with the highest correlation with spot 1, was Der p 1 allergen. FIG. 2B shows that spots 1 and 2 were selected for LC MS/MS analysis, and peptide sequence ions from the N-terminus and C-terminus are indicated.

FIGS. 3A-3B depict the epitope mapping of antibodies which bind Der p 1 precursor amino acid sequences was via an ELISA analysis, and the three-dimensional structure of Der p 1 shows the active site and the epitope site. FIG. 3A shows the peptide map of the Der p 1 precursor was predicted by computer. FIG. 3B shows the two peptide fragments with the strongest binding to mAb W108.

FIGS. 4A-4D depict the effect of mAb W108 on Der p-sensitized and allergen challenged mice. FIG. 4A shows airway resistance induced by various dosage of methacholine and measured by PenH. FIG. 4B shows the anti-Der p IgG subclass and IgE antibodies in sera. FIG. 4C shows the differential cell counts in bronchoalveolar fluid. FIG. 4D shows cytokines production in bronchoalveolar fluid.

FIGS. 5A-5B depict the effect of mAb W108 on Der p-sensitized and allergen challenged mice. FIG. 5A shows airway resistance induced by various dosage of methacholine and measured by PenH. FIG. 5B shows anti-Der p IgG subclass and IgE antibodies in sera.

### DETAILED DESCRIPTION OF THE INVENTION

The following abbreviations are used throughout the present invention:

Der p 1=Dermatophagoid pteronyssinus;

CDR=complementary determining regions;

mAb=monoclonal antibody;

IgE=immunoglobulin E;

BALF=Bronchoalveolar lavage fluids;

PBS=phosphate buffered saline;

2-DE=Two-dimensional electrophoresis;

a.a.=amino acids.

Unless defined otherwise, all technical and scientific terms used herein have the same meanings as those commonly understood to one of ordinary skill in the art to which this invention pertains.

As used herein, the singular forms “a”, “an”, and “the” include plural referents unless the context clearly dictates otherwise. Thus, for example, reference to “a sample” includes a plurality of such samples and equivalents thereof known to those skilled in the art.

As used herein, the term “antibody” refers to polyclonal antibodies, monoclonal antibodies, humanized antibodies, single-chain antibodies, and fragments thereof such as F_{ab}, F_{(ab′)2}, Fᵥ, and other fragments which retain the antigen binding function of the parent antibody.

As used herein, the term “monoclonal antibody” refers to an antibody composition having a homogeneous antibody population. The term includes but is not limited to the species or source of the antibody, nor is it intended to be limited by the manner in which it is made. The term encompasses whole immunoglobulins as well as fragments such as F_{ab}, F_{(ab′)2}, Fᵥ, and others that can bind to the antigen of the antibody. Monoclonal antibodies of any mammalian species can be used in this invention.

As used herein, the term “humanized antibodies” means that at least a portion of the framework regions of an immunoglobulin are derived from human immunoglobulin sequences.

As used herein, the term “IgE-mediated disease” refers to a disease mediated by IgE, which includes but is not be limited to allergic rhinitis or asthma.

The present invention provides an isolated peptide segment which is a Dermatophagoid pteronyssinus (Der p1) antigen epitope, comprising one selected from the group consisting of the peptide segments having the amino acid sequences set forth in SEQ ID NO: 3 and SEQ ID NO: 4, and combination thereof It is found in the invention that the Dermatophagoid pteronyssinus (Der p1) antigen epitope can induce air way inflammation.

In one certain embodiment, Native Der p 1 was purified from whole culture extracts of Der p. 6-week-old female BALB/c mice were used for immunization with Der p extracts and 8-week-old female BALB/c mice were used for production of ascites. For the production of mAbs, female BALB/c mice were immunized with Der p extracts. The mouse with the highest antibody titer was injected with antigen. The spleen cells of animals were fused with myeloma cells. Supernatants from the fused cells were screened for anti-Der p-specific antibodies by ELISA with purified native Der p 1 in the solid phase. The positive hybrids were cloned and sub-cloned by limiting dilution. The isotype of the antibodies was determined by ELISA with anti-mouse subclass antisera and the mAb was purified from hybridoma supernatants using standard protein G affinity chromatography.

On the other hand, the invention provides an isolated antibody or antigen binding fragment thereof specific for a Dermatophagoid pteronyssinus (Der p1) antigen, which binds to the peptide segment of Dermatophagoid pteronyssinus (Der p1) antigen epitope. Particularly, an isolated antibody or antigen binding fragment comprises a light chain variable region comprising one amino acid sequence set forth in SEQ ID NO: 1 and a heavy chain variable region comprising one amino acid sequence set forth in SEQ ID NO: 2. The antibody of the present invention may be a polyclonal or monoclonal antibody, which has the capacity to inhibit Der p 1 allergen-induced inflammation In a preferred embodiment of the invention, the antibody is a monoclonal antibody.

In another embodiment, the inhibition of the binding of specific IgE to Der p extracts and/or native Der p 1 by mAb was evaluated by competitive inhibition in a modified ELISA. It is confirmed in the invention that the antibody has activity to block the binding of allergen-specific IgE in individual and pooled sera from nine Der p-sensitive allergic asthmatic children.

Specifically, the Der p 1 protein was suspended in sample solution and 2-DE was performed. Bound monoclonal antibodies were detected using peroxidase-labeled goat anti-mouse IgG antibodies. Protein spots of interest were removed from the 2-D gel and transferred to tubes for in-gel digestion, and directly spotted onto matrix-assisted laser desorption plates for liquid chromatography tandem mass spectrometry analysis.

As used herein, the overlapping peptide fragments of Der p 1 were amplified by mutiplex PCR methods. Gel-purified PCR products were ligated into pGEX-2T vector. The peptides were expressed as glutathione S-transferase fusion protein/peptides in E. coli by isopropyl-B-D-thiogalactopyranoside induction. The purification of recombinant proteins was obtained by affinity chromatography using a glutathione-Sepharose column.

According to the invention, total mRNA was extracted from Der p-specific IgG2b-producing hybridoma clones. PCR amplification was carried out using cDNA in each reaction, and the primers to amplify the complete kappa chain cDNA. These PCR products were cloned into the vector pCRTM. The amino acid sequence of CDR3 of the heavy chains (SEQ ID NO: 2) as determined by automatic amino acid sequencing. Similarly, the light chain included the amino acid sequence of CDR3 (SEQ ID NO: 1) in the light chain of mAb.

In another one certain embodiment, the airway resistance of mice was measured in a single-chamber. Bronchoalveolar fluids (BALF) of treated and non-treated mice were centrifuged to count infiltrating cells, and supernatants were collected for cytokine analysis. Inflammatory cell infiltrates and lung architecture were assessed using light microscopy. Levels of the cytokines, IL-4, IL-5, IFN-g and Eotaxin, in the BALF were assayed in ELISA kits.

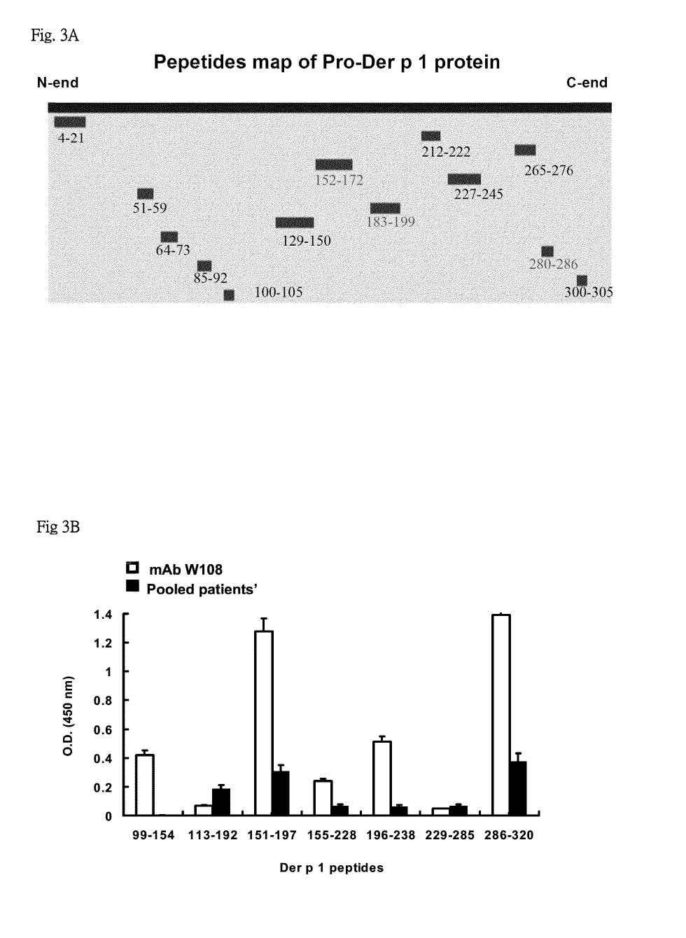

According to the invention, an isolated monoclonal antibody inhibited the binding between human anti-Der p-specific IgE antibody and Der p 1. Moreover, mAb also inhibited the cysteine protease activity of Der p 1, Der p 1 allergen-induced airway inflammation and other immunological changes in the mouse model of asthma. mAb shared epitope specificity with human anti-Der p 1 IgE. The epitopes on the Der p 1 which bind IgE may not be continuous in nature which depended on the presence of conformational IgE epitopes. The two peptide segments of Der p 1 (SEQ ID NOs: 3 and/or 4) which bind to mAb as shown in FIG. 3A-FIG. 3B are parts of connecting loops located in the substrate-binding cleft and on the surface of domain consisting mainly of β-sheets. The interaction of the amino acid sequence in the CDR3 of mAb (SEQ ID NO: 1 and/or 2) with Der p 1-binding epitopes (SEQ ID NOs: 3 and/or 4) containing the active site of cysteine protease activity. mAb-binding epitopes are protruding loops, therefore, it is capable of inhibiting IgE binding to Der p 1 antibody and cysteine protease activity through steric hindrance. The anti-Der p 1 mAb attenuated Der p allergen-induced airway inflammation and the allergic immune response in mouse model of allergic asthma.

The present invention also provides a method for preventing and/or treating an IgE-mediated disease in a subject comprising administering the subject with a therapeutically effective amount of the antibody or antigen binding fragment specific for Dermatophagoid pteronyssinus (Der p1) antigen.

In one embodiment, the monoclonal antibody or antigen binding fragment of the present invention can be incorporated into pharmaceutical compositions suitable for administration to a subject. Typically, the pharmaceutical composition for treating an IgE-mediated disease comprising a therapeutically effective amount of the antibody or antigen binding fragment of present invention.

In another embodiment, the isolated peptide segment of Dermatophagoid pteronyssinus (Der p1) antigen epitope can be incorporated into vaccine composition suitable for administration to a subject. Typically, vaccine composition for preventing IgE-mediated disease comprising administering the subject with a therapeutically effective amount of the peptide segment of Dermatophagoid pteronyssinus (Der p1) antigen epitope. Moreover, the vaccine composition further comprises a vaccine adjuvant.

The present invention is further illustrated by the following examples, which are provided for the purpose of demonstration rather than limitation.

### Example 1

### Generation of Anti-Der p 1 Monoclonal Antibody of the Invention

1.1 Preparation of Der p 1 Extracts

Der p extracts from Pharmacia Allergon, Inc;(Sweden) were prepared following the manufacturer's instructions and stored at −80° C. before use. Native Der p 1 was purified from whole culture extracts of Der p 1.

1.2 Human Sera

Sera from Der p-sensitive patients, collected in the Allergic Clinic of National Cheng Kung University Hospital. The allergic phenotype was confirmed by clinical history, skin prick tests, and a high level of IgE reactivity against Der p in UniCAP tests (Pharmacia Diagnostics, UK). A panel of 10 Der p extract-positive sera from asthmatic and/or atopic dermatitis patients was used for IgE ELISA, and 10 sera from individuals who were not sensitive to any inhalant allergens were used as negative controls. All sera were stored at −80° C. until use.

1.3 preparation of anti-Der p 1 mAb

Five 6-week-old female BALB/c mice were used for immunization with Der p extracts and thirty 8-week-old female BALB/c mice were used for production of ascites. For the production of mAbs, female BALB/c mice were immunized with Der p extracts. Blood was collected from the infra-orbital plexus every week to monitor the titer of Der p-specific antibodies by ELISA. Three days before fusion, the mouse with the highest antibody titer was injected with 100 g of antigen in PBS. This animal was killed and its spleen cells were fused with FO-mouse myeloma cells. Ten days after the fusion, culture supernatants from the fused cells were screened for anti-Der p-specific antibodies by ELISA with purified native Der p 1 in the solid phase. The positive hybrids were cloned and sub-cloned by limiting dilution. The mAb W108 was purified from hybridoma culture supernatants using standard protein G affinity chromatography (Sigma-Aldrich, USA). The isotype of the antibodies was determined by ELISA with anti-mouse subclass antisera (Nordic, Tilburg, Netherlands).

### Example 2

### mAb W108 Inhibited Der p-specific IgE Binding and Protease Activity of Der p 1 of the Invention

2.1 Inhibition Assay

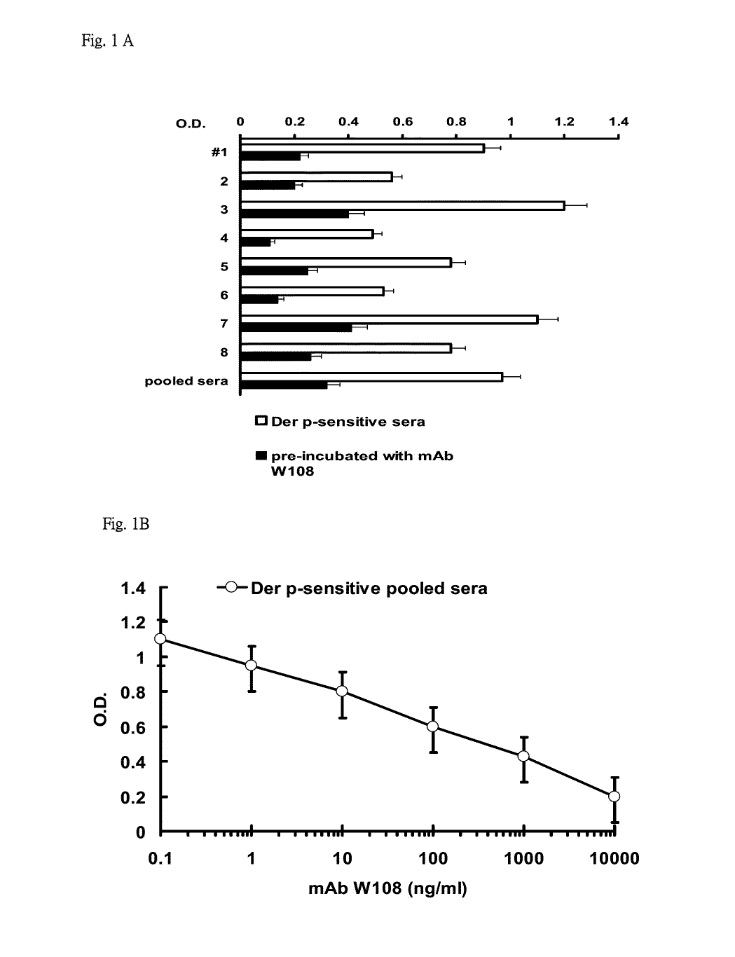

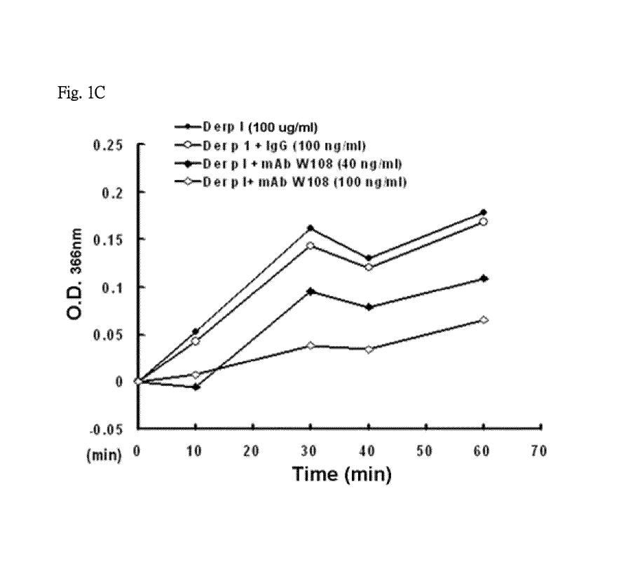

The inhibition of the binding of specific IgE to Der p extracts and/or native Der p 1 by mAb W108 was evaluated by competitive inhibition in a modified ELISA. Detection of a bound human specific IgE antibody was performed using HRP-conjugated mouse anti-human IgE Ab (Zymed, USA) as shown in FIG. 1A-FIG. 1C.

2.2 Results

mAb W108 (selected from over 700 positive antibodies) had the highest blocking activity (i.e., inhibited >70% of the binding of allergen-specific IgE in individual and pooled sera from nine Der p-sensitive allergic asthmatic children, FIG. 1A and FIG. 1B). Western blot analysis showed that mAb W108 blocked the binding of serum Der p-specific IgE to 36-kDa bands in the Der p extract. Using ELISA plates coated with purified native Der p 1, mAb W108 was found to dose-dependently inhibit serum Der p-specific IgE binding to Der p 1. Using azocasein as a substrate, mAb W108 inhibited not only the binding of Der p 1, but also its cysteine protease activity in a dose-dependent manner (FIG. 1C).

### Example 3

### Proteomic Analysis of Der p Extracts with Pooled Sera from Der p-Sensitized Patients and mAb W108 of the Invention

3.1 2-DE Immune-Detecting and Proteomic Analysis of the Invention

The Der p protein was suspended in 400 μl of sample solution (7 M urea, 2 M thiourea, 2% CHAPS, 0.5% IPG buffer [immobilized pH gradient], pH 4-7, 0.003% bromophenol blue) and 2-DE was performed. Bound monoclonal antibodies were detected using peroxidase-labeled goat anti-mouse IgG antibodies (Zymed). Protein spots of interest were removed from the 2-D gel and transferred to 650-μl tubes (siliconized) for in-gel digestion, and directly spotted onto MALDI plates for liquid chromatography tandem mass spectrometry analysis. Mass analyses were undertaken using the PerSeptive Biosystems Voyager-DE STR MALDI-TOF mass spectrometer (Framingham, Danvers, Mass.). Peptide mapping was performed by comparison with the deduced amino acid sequence of Der p 1 cDNA. The amino acid sequence was translated from proteomic data using the MASCOT Tool from Matrix Science (http://www.matrixscience.com). Homology search of the predicted amino acid sequence was conducted using the BLAST network server at the National Center for Biotechnology Information (http://www.ncbi.nlm.nih.gov).

3.2 Results

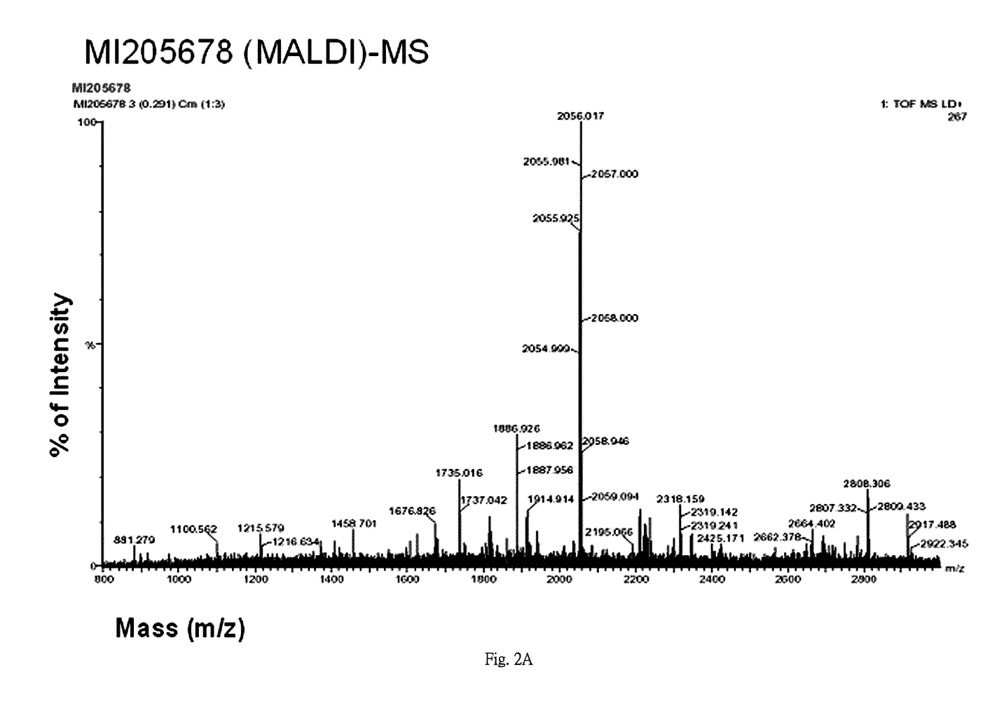

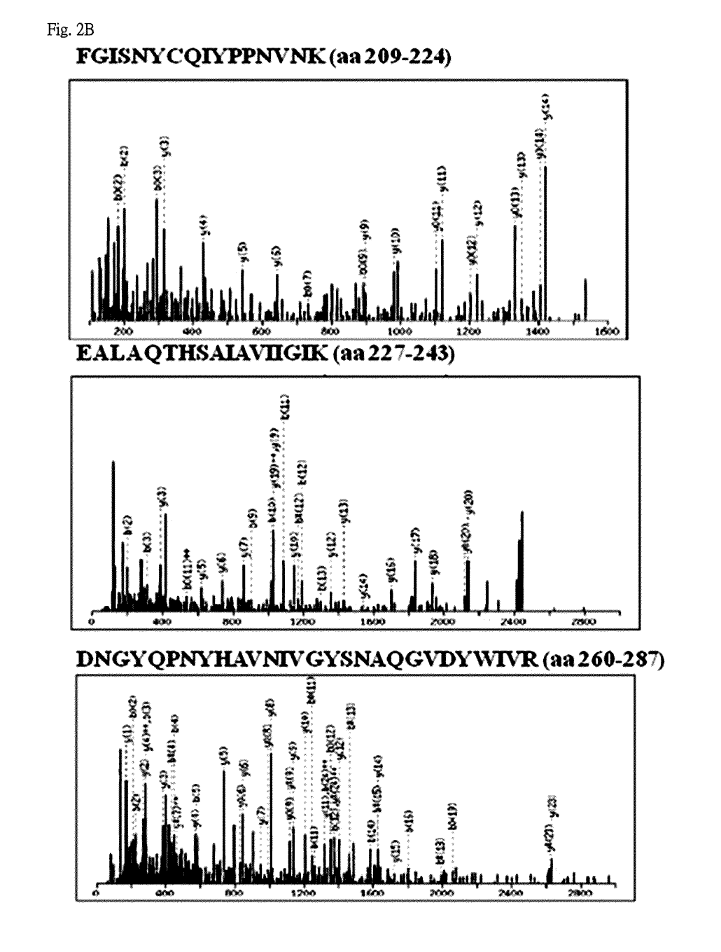

A total of over 200 distinct spots were detected after silver staining and image analysis (BioRad PDQuest software) in 2D gel electrophoresis. Replicate gels gave spots reproducibility ranging between 86 and 91% (n=5 replicates each). Using a silver stain, approximately 275 distinct and reproducible spots in Der p extract were detected. Immuno-blotting showed that mAb W108 reacted with four components of Der p extracts with a molecular mass of 36 kDa and pI values varying from 5 to 6. These spots also reacted to pooled sera from Der p-sensitive subjects. In inhibition studies, specific binding to these protein spots by IgE in pooled sera from Der p-sensitive patients was blocked by pre-incubation with mAb W108. All four of these spots were then excised and subjected to in situ in-gel trypsin digestion for MALDI-TOF MS analysis. The MS profile showed multiple peaks in the range of 800-2800 Da (FIG. 2A). One peak with molecular mass 2056.0 Da was selected for comparison with established databases, and the proteins with the highest correlation with the analyzed spot were the cysteine protease from Der p allergen (accession number gi1460058, score 162) and the major mite fecal allergen Der p 1 precursor (accession number gi730036, score 121), with sequence coverage of nearly 99%.

The 36-kDa spots (pI 5-6) had identical amino acid sequences, which showed their identity with the known partial sequence of the Der p 1 isoform 3 pre-cursor. When the internal sequences of the mAb were further characterized, W108 recognized Der p peptides were selected for LC MS/MS analysis, and the generated fragment-ion spectrum identified these two peptides as having the same sequences—a.a. 209-224, a.a. 227-243 and a.a. 260-287. All sequences of these fragments were identical with the Der p 1 precursor sequence.

Example 4

### Identification of Epitopes on Der p 1 Recognized by mAb W108

4.1Recombinant Der p 1 Peptide

The overlapping peptide fragments of Der p 1 were amplified by mutiplex PCR methods. Gel-purified PCR products were ligated into pGEX-2T vector (Pharmacia Biotech, UK). The peptides were expressed as glutathione S-transferase fusion protein/peptides in E. coli by isopropyl-B-D-thiogalactopyranoside induction. The purification of recombinant proteins was obtained by affinity chromatography using a glutathione-Sepharose column (Sigma, USA)

4.2 Results

To identify the linear mAb-binding epitopes on Der p 1 allergen recognized by mAb W108, peptides were constructed to cover the sequences of the IgE-binding epitopes of Der p 1, and predicted by the computer software (FIG. 3A). To test the binding of mAb W108 to these peptides, 7 peptide fragments, covering almost the entire amino acid sequence of Der p 1, were screened by solid phase ELISA. Two peptide fragments (a.a.151-197 and 286-320) showed the strongest binding to mAb W108 (FIG. 3B) and were highly bound to specific-IgE antibodies from the pooled sera (n=10) of Der p-sensitized patients (FIG. 3B).

Example 5

### Identification of Heavy and Light Chain Paratopes in Complementarity Determining Regions (CDR) of mAb W108

5.1 RT-PCR amplification from mAb W108 clone

Approximately 1×10⁶ W108 hybridoma cells were harvested by centrifugation, and homogenized. Total RNA was prepared using the standard Trizol™ kit (Invitrogen, USA). P PCR amplification was carried out using cDNA in each reaction, and the primers to amplify the complete kappa chain cDNA.

5.2 Molecular Modeling

Using the 3D structure of the major house dust mite allergen Der p 1 (PDB accession number 1×kg), a model structure of Der p 1 was constructed using the SWISS-MODEL program (http://swissmodel.expasy.org/SWISS-MODEL.html). The solvent-accessible surface area of individual amino acids was calculated using the GETAREA program (www.scsb.utmb.edu/cgi-bin/get-a-form.tel). This program considers residues as buried if the ratio of the side-chain surface area to a random coli reference value per residue is <20% and the solvent as exposed if the ratio exceeds 50%. The solvent-accessible surface area on the mature protein covered by the peptide was calculated using the CCP4 program ArealMol. All structural images were prepared with the program pymol (http://www.pymol.org).

5.3 Results

These PCR products were cloned into the vector pCRTM 2.1. The amino acid sequence of CDR3 of the heavy chains (SEQ ID NO: 2) as determined by automatic amino acid sequencing. Similarly, the light chain included the amino acid sequence of CDR3 (SEQ ID NO: 1) in the light chain of mAb W108. These data indicate that the W108 mAb is a novel anti Der p 1 monoclonal antibody, which is also valuable for clinical applications.

These PCR products were cloned into the vector pCRTM 2.1, and 10 clones from each reaction were selected for an amino acid sequence analysis of the cloned plasmids. The first eight amino acids of the light chain and the first nine amino acids of the heavy chain were derived from the degenerate PCR primers. The amino acid sequences for the CDRs in the light and heavy chains of mAb W108 are SEQ ID NO: 1 and 2. The V-region protein sequences in the heavy and light chains of mAb W108 were compared with the homologous sequences in the V-BASE database, and a number of homologous V-genes were identified. The heavy chain of mAb W108 had a >90% homology with a member of the VH3 family. The amino acid sequence of CDR3 of the heavy chains was SEQ ID NO: 2 (a.a. 105-115) as determined by automatic amino acid sequencing. Similarly, the light chain had a >80% homology with M15520 IGKV10-96*01, including the predicted CDR1 and CDR2, and the amino acid sequence of CDR3 was SEQ ID NO: 1 (aa. 105-115) in the light chain of mAb W108.

### Example 6

### mAb W108 Inhibits Der p-Induced Airway Inflammation

6.1 Immunization of Mice

The airway resistance of mice was measured in a single-chamber, whole-body plethysmograph (Buxco Electronics, Inc, Troy, N.Y.). Bronchoalveolar fluids (BALF) of treated and non-treated mice were centrifuged to count infiltrating cells, and supernatants were collected for cytokine analysis. Inflammatory cell infiltrates and lung architecture were assessed using light microscopy. Levels of the cytokines, IL-4, IL-5, IFN-y and Eotaxin, in the BALF were assayed in ELISA kits (R&D Diagnostics, USA).

6.2 Results

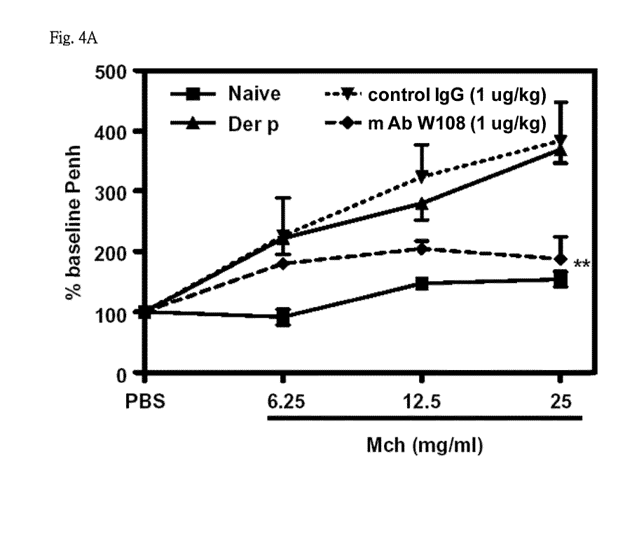

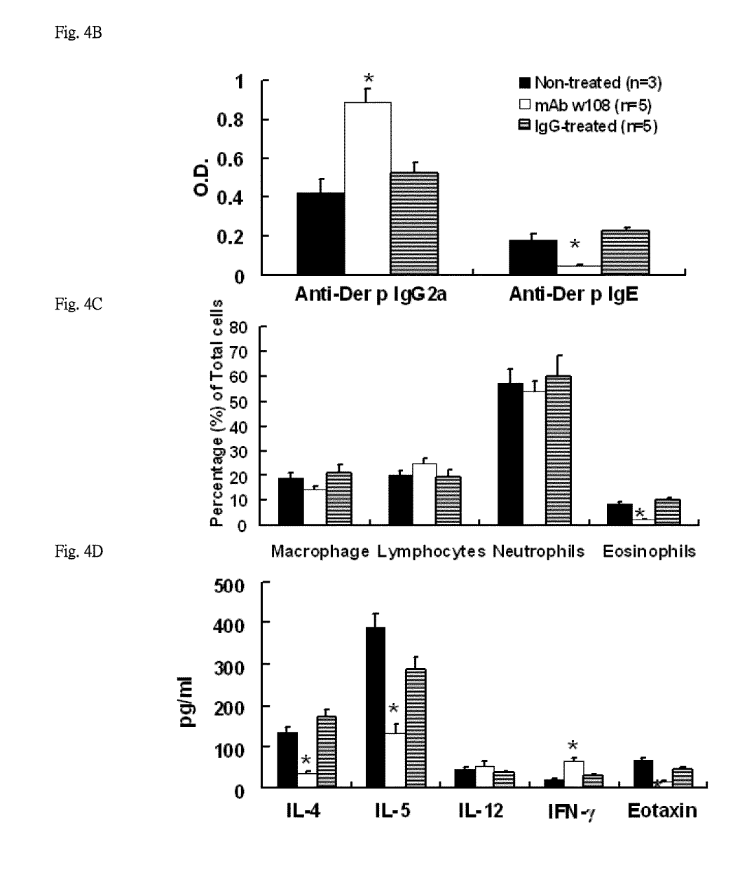

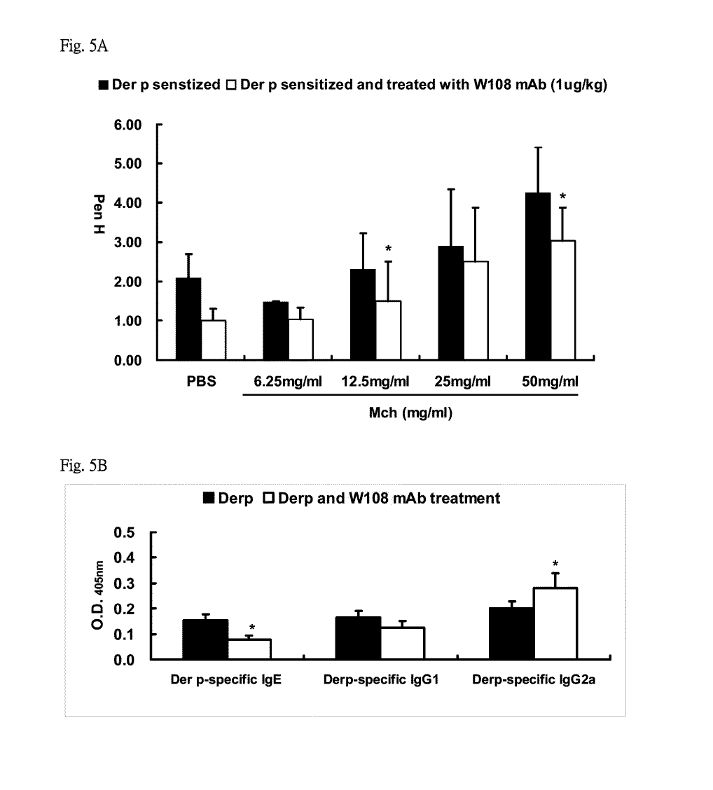

The in vivo effect of mAb W108 on the Der p-induced airway inflammation was studied in Der p-sensitized mice. Weekly doses of mAb W108 (1 μg/kg) injected during allergen sensitization for 2 weeks and 6 hours before challenge attenuated airway inflammation, lower airway hyperreactivity (FIG. 4A), titer of Der p-specific IgE in serum, eosinophil infiltration , IL-4, IL-5, and the production of eotaxin in BALF. In contrast, Der p-specific IgG2a in serum and INF-y in BALF were increased in mAb W108-treated mice as compared with non-treated mice or mice sensitized with non-specific IgG. There were similar inhibitory effect of mAb W108 on Der p-induced airway inflammation, airway hpereactivity (FIG. 5A), Der p-specific IgE production (FIG. 5B).

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. An isolated antibody or antigen binding fragment thereof specific for a Dermatophagoid pteronyssinus (Der p1) antigen, which binds a Dermatophagoid pteronyssinus (Der p1) antigen epitope, comprising one selected from the group consisting of the peptide segments having the amino acid sequences set forth in SEQ ID NO: 3 and SEQ ID NO: 4, and combination thereof.

2. The antibody or antigen binding fragment according to claim 1, wherein said antibody or antigen binding fragment comprises a light chain variable region comprising one amino acid sequence set forth in SEQ ID NO: 1.

3. The antibody or antigen binding fragment according to claim 1, wherein said antibody or antigen binding fragment comprises a heavy chain variable region comprising one amino acid sequence set forth in SEQ ID NO: 2.

4. The antibody or antigen binding fragment according to claim 1, which is a monoclonal antibody.

5. The antibody according to claim 4, having a light chain variable region comprising one amino acid sequence set forth in SEQ ID NO: 1 and a heavy chain variable region comprising one amino acid sequence set forth in SEQ ID NO: 2.

6. The antibody or antigen binding fragment according to claim 1, having the capacity to inhibit Der p 1 allergen-induced inflammation.

7. A method for preventing and/or treating an IgE-mediated disease in a subject comprising administering the subject with a therapeutically effective amount of the antibody or antigen binding fragment according to claim 1.

8. A method for preventing and/or treating an IgE-mediated disease in a subject comprising administering the subject with a therapeutically effective amount of the antibody or antigen binding fragment according to claim 2.

9. A method for preventing and/or treating an IgE-mediated disease in a subject comprising administering the subject with a therapeutically effective amount of the antibody or antigen binding fragment according to claim 3.

10. The method according to claim 7, comprising administering the subject with a therapeutically effective amount of the antibody according to claim 5.

11. The method according to claim 10, wherein said IgE-mediated disease is allergic rhinitis or asthma.

12. A pharmaceutical composition for preventing and/or treating an IgE-mediated disease comprising a therapeutically effective amount of the antibody or antigen binding fragment according to claim 1.

13. A pharmaceutical composition for preventing and/or treating an IgE-mediated disease comprising a therapeutically effective amount of the antibody or antigen binding fragment according to claim 2.

14. A pharmaceutical composition for preventing and/or treating an IgE-mediated disease comprising a therapeutically effective amount of the antibody or antigen binding fragment according to claim 3.

15. The pharmaceutical for preventing and/or treating an IgE-mediated disease comprising a therapeutically effective amount of the antibody according to claim 5.
